(19) United States
(12) Patent Application Publication
(10) Pub. No.: US 2013/0205880 A1
(43) Pub. Date: Aug. 15, 2013

(54) TILTING SYSTEM AND METHOD FOR TESTING ENGINE LUBRICANTS

(71) Applicant: Abraham Robert DE KRAKER, Sugar Land, TX (US)

(72) Inventor: Abraham Robert DE KRAKER, Sugar Land, TX (US)

(73) Assignee: SHELL OIL COMPANY, Houston, TX (US)

(21) Appl. No.: 13/599,941

(22) Filed: Aug. 30, 2012

(51) Int. Cl. G01N 33/28

(52) U.S. Cl. CPC G01N 33/2888
USPC 73/114.55

(57) ABSTRACT

Systems and methods for testing an engine lubricant are provided. The system includes a tilting platform selectively movable between tilt angles and at least one test tray. The tilting platform includes a heated block with a heat source. The test tray has the engine lubricant therein. The test tray also has a heated portion and an unheated portion. The test tray is tiltably positionable on the tilting platform with the heated portion heatable by the heated block whereby the engine lubricant is selectively positionable about the heated and unheated portions of the test tray.

### BACKGROUND

The present disclosure relates generally to testing operations. More specifically, the present disclosure relates to techniques for testing fluids, such as lubricants used in engines.

Lubricants may be used in machinery to prevent friction between moving parts, such as pistons and cylinders of an engine. In some cases, deposits may form in the lubricants that may hinder the movement of the parts and, therefore, impact the performance of the engine. Lubricants may be configured to reduce the likelihood of deposits and/or affect the performance of the engine.

Lubricants may include a mix of oils and other additives. The composition of the lubricant may be selected to define properties which can be used to enhance performance of the machinery. For example, various engines may specify the use of a certain viscosity of lubricant under certain conditions, such as outdoor temperature. In another example, the composition of the lubricant (and/or its additives) may be selected to control the engine's tendency to oxidize and form deposits.

Designed experiments may be performed to compare lubricants having various compositions. The experiments may involve performing tests of various lubricants to determine how each lubricant will perform in an engine. The experiments may be performed using apparatuses that simulate the engine and provide controlled conditions for testing. Examples of tests are provided in U.S. Pat. Nos. 5,313,824, 5,287,731, 7,597,016, 6,571,611, 6,566,142 and 5,492,005.

In some cases, experiments may be conducted to determine properties of different lubricants which may affect the performance of the machinery. For example, tests may be performed to determine oxidation of lubricants. Examples of oxidation tests include TFOUT (Thin-Film Oxygen Uptake Test), PDSC (Pressurized Differential Scanning calorimetry), Ciba Viscosity Increase Test (CVIT), HOOT (Hot Oil Oxidation Test), FOAT (Ford Oil Aging Test), and Oxidator (Oronite Oxidation) test. Tests may also be performed to detect deposit formation. Examples of deposit tests include inclined plane, panel coker, hot tube, sliding ring, and micro-oxidation. Facilities used in performing the various tests may be configured to simulate environments in which the lubricants are used.

### SUMMARY

In at least one aspect, the disclosure relates to a system for testing an engine lubricant. The system includes a tilting platform and at least one test tray. The tilting platform is selectively movable between tilt angles. The tilting platform includes a heated block with a heat source. The test tray has the engine lubricant therein. The test tray has a heated portion and an unheated portion. The test tray is tiltably positionable on the tilting platform with the heated portion heatable by the heated block whereby the engine lubricant is selectively positionable about the heated and unheated portions of the test tray.

The tilting platform may include a base portion and a tilting portion pivotally connectable by a hinge. The tilting platform may include a pair of supports with a support arm operatively connecting the heated block thereto. The heated block may be positionable on the tilting platform and movable thereby. The tilting platform may include a test block positionable on the heated block and heatable thereby. The heated block may include a frame with a heated bar and an unheated bar operatively connectable thereto. The heated portion may be positioned on the heated bar and the unheated portion may be positioned on the unheated bar. The system may also include a motor to selectively move the tilting platform between the tilt angles. The motor may include a piston and cylinder. The piston may be selectively extendable from the cylinder to move the tilting platform between tilting angles. The motor may include a rotational driver. The test tray may include a plurality of test dishes and/or a dish tray having a plurality of test cups.

In another aspect, the disclosure relates to a system for testing an engine lubricant. The system includes a tilting platform selectively movable between tilt angles. The tilting platform includes at least one test block heatable by a heat source, at least one test tray and a driver. The test tray has the engine lubricant therein. The test tray has a heated portion and an unheated portion. The test tray is tiltably positionable on the tilting platform with the heated portion on the heated block whereby the engine lubricant is selectively positionable about the heated and unheated portions of the test tray. The driver is operatively connectable to the tilting platform and selectively moving the tilting platform between tilt angles.

Finally in another aspect, the disclosure relates to a method for testing an engine lubricant. The method involves providing a system for testing the engine lubricant. The system includes a tilting platform and at least one test tray. The tilting platform includes a heated block with a heat source. Each test tray has the engine lubricant therein. Each test tray has a heated portion and an unheated portion. The method further involves heating the heated portion with the heat block, positioning the test trays on the tilting platform, selectively positioning the engine lubricant about the heated and unheated portions of the test tray by selectively moving the tilting platform between tilt angles, and examining the test trays for deposits. The selectively positioning may also involve positioning an unheated portion of the test tray about an unheated block of the heated tilting platform. The selectively moving may involve pivotally moving the platform and/or rotationally moving the platform.

### BRIEF DESCRIPTION OF THE DRAWINGS

So that the above recited features and advantages of the disclosure may be understood in detail, a more particular description of the disclosure, briefly summarized above, may be had by reference to the embodiments thereof that are illustrated in the appended drawings. It is to be noted, however, that the appended drawings illustrate only typical embodiments of this disclosure and are, therefore, not to be considered limiting of its scope. The figures are not necessarily to scale, and certain features and certain views of the figures may be shown exaggerated in scale or in schematic in the interest of clarity and conciseness.

FIGS. 1A and 1B are schematic diagrams depicting a system for testing engine lubricants at a first tilt angle and a second tilt angle, respectively, in accordance with the present disclosure;

FIGS. 2A1-2A4 and 2B are schematic diagrams depicting test trays in accordance with the present disclosure;

FIGS. 3A and 3B are schematic diagrams depicting another system for testing engine lubricants at a first tilt angle and a second tilt angle, respectively, in accordance with the present disclosure; and

FIG. 4 is a flow chart depicting a method for testing engine lubricants in accordance with the present disclosure.

### DETAILED DESCRIPTION

The description that follows includes exemplary apparatuses, methods, techniques, and instruction sequences that embody techniques of the inventive subject matter. However, it is understood that the described embodiments may be practiced without these specific details.

The disclosure relates to techniques for testing lubricants, such as those used in engines. These techniques may involve the use of a test tray tiltable such that testing lubricant therein is selectively positionable on heated and unheated portions of the test tray. The test trays may be examined for the formation of deposits. The experiments may be performed over time and selectively controlled to define a test cycle. The system is configured to incorporate characteristics of a combustion engine, such as an oil sump at adjustable temperature with a hot surface to simulate a piston at a secondary, higher temperature. The test simulates an engine by moving oil from a lower temperature sump to a higher temperature engine piston, thereby creating deposits.

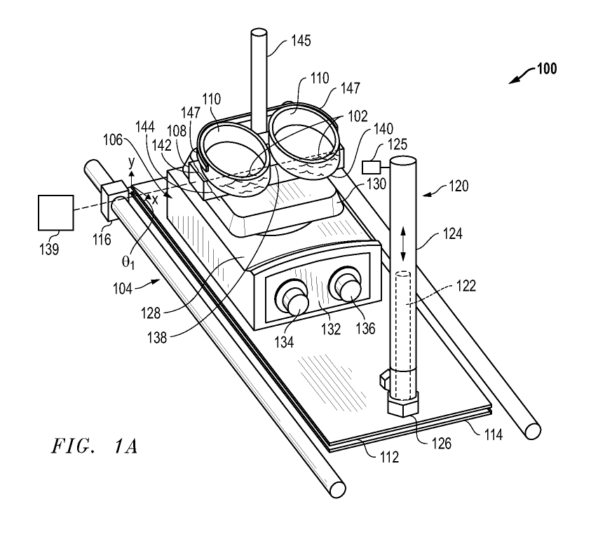

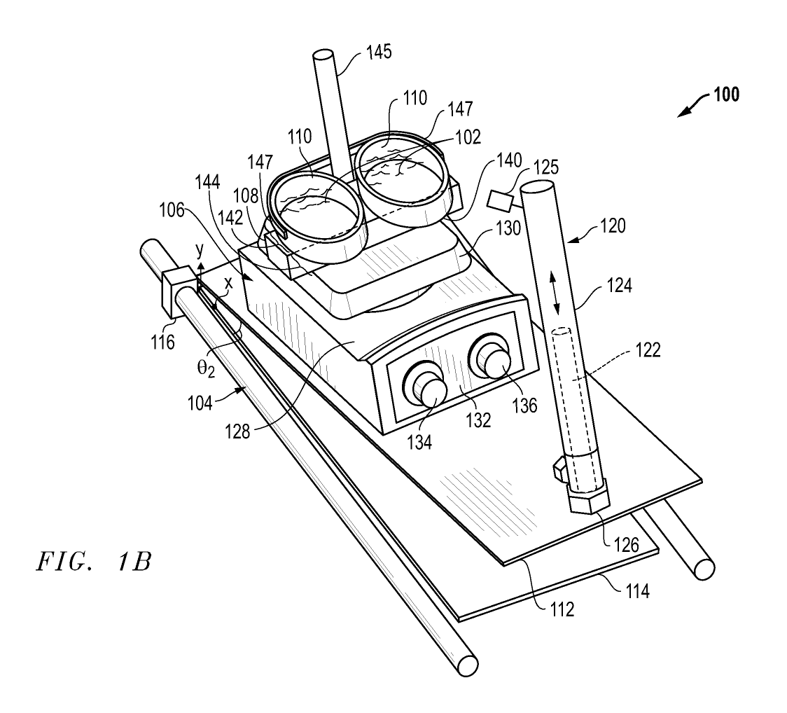

FIGS. 1A and 1B illustrate a test system 100 usable for evaluating a lubricant 102. FIG. 1A shows the test system 100 at a first tilt angle θ₁. FIG. 1B shows the test system moved to a second tilt angle θ₂. The system 100 includes a platform 104, a heated block 106, a test block 108 and test trays 110. The platform 104 includes a support 112 tiltable about a surface. In the version of FIG. 1, the support 112 is pivotally connected to a base 114 by a hinge 116. The base 114 may be positioned upon a surface (e.g., countertop) and remain stationary during the test. The support 112 may tilt up and down at a tilting angle θ relative to the base 114.

The platform 104 is depicted as also having a driver 120 connected thereto. The driver 120 includes a piston 122 and a cylinder 124. A far end 126 of the piston 122 is fixedly connected to the base 114. The cylinder 124 has the piston 122 slidably positionable therein and is fixedly connected to the support 112. The piston 122 is selectively extendable from the cylinder 124. As the piston 122 is extended, the cylinder 124 and the support 112 connected thereto are lifted to the tilting angle θ. Other configurations may be provided for tilting the platform 104 to a desired tilting angle θ as will be described herein.

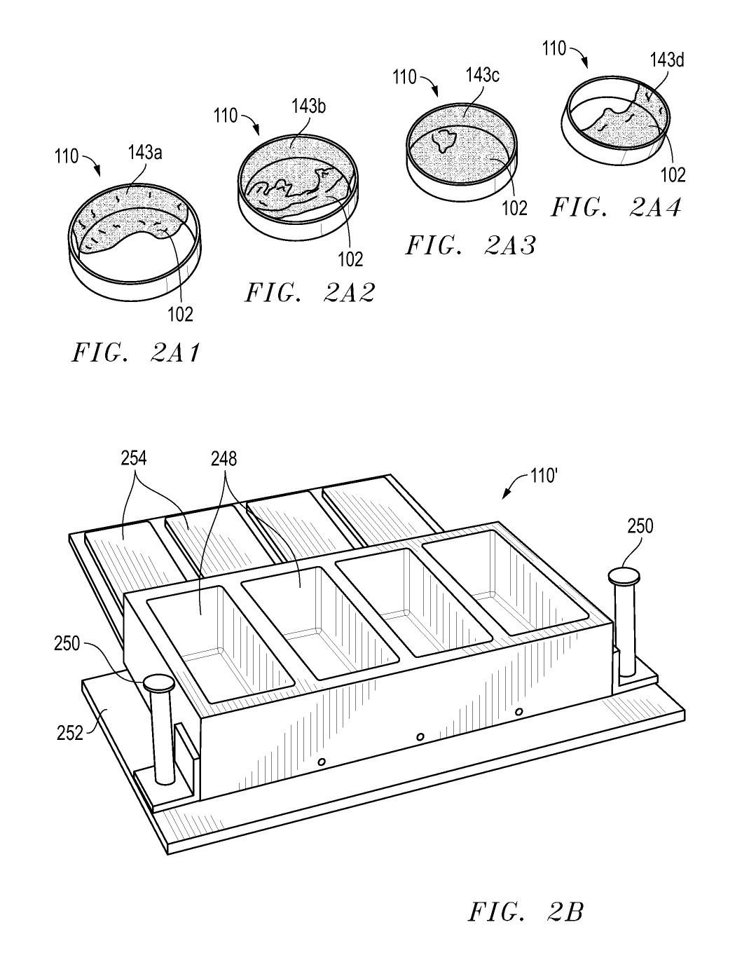

A tilt controller 125 is operatively connectable to the platform 104 and/or the driver 120. The tilt controller 125 may be used to selectively activate the driver 120 to move the platform 104 between tilt angles, such as tilt angle θ₁ of FIG. 1A and tilt angle θ₂ of FIG. 2A. The tilt controller 125 may provide the desired movement to the desired tilt angle and the desired timing as desired. The tilt controller 125 may be provided with a processor to monitor the test and feedback information to activate the tilt controller 125.

The heated block 106 includes a frame 128 and a heat plate 130. The heat plate 130 may be a conventional heating device, such as an electrical heater. The heat plate 130 has a heat controller 132 for providing a desired temperature. The heat controller 132 has knobs 134, 136 for selecting the desired temperature and time, respectively. The heat controller 132 may be a programmable controller (e.g., with a processor or linked to a processor) for providing heat as desired. The heat may be applied at a given temperature and for a given time frame in one or more cycles.

The test block 108 may be a metal component with sufficient conductivity to conduct a desired amount of heat from the heat block 106 to the test trays 110. The test block 108 has a test surface 138 for supporting one or more test trays 110. The test surface 138 is shown as a flat, rectangular surface, but may be of any dimension sufficient for supporting the test trays 110 and applying heat to a heated portion 142 thereof. The test surface 138 may have a series of troughs therein which may be connected, for example, to a vacuum source, the vacuum holding the test trays 110 on the test surface 138.

The test trays 110 are depicted as shallow cups for receiving the test lubricant 102. Two test trays 110 are shown, but one or more may be used. The test trays 110 have a bottom 140 positionable on the test block 108. The heated portion 142 of the test tray 110 rests on the test block 108 and receives heat therefrom. An unheated (or ambient) portion 144 of the test tray 110 extends beyond the test block 108 and is not heated thereby.

The test trays 110 may be secured to the test block 108 by a support arm 145. The support arm 145 rests on the platform 104 and extends vertically thereabove. The support arm 145 has grips 147 for grippingly holding the test trays in position on the test block 108. The support arm 145 is movable with the platform 104 as it tilts to maintain the test trays 110 in position above the test block 108.

The test lubricant 102 may be any lubricant (e.g., motor oil) or mix of lubricants to be tested. A mixture of lubricants may include the test lubricant 102 and another lubricant, such as a taxi oil. A taxi oil refers to lubricants that have been previously used over a period of time, for example, in a taxi cab. The taxi oil may be added in desired ratios with the test lubricant to speed up the oxidation and/or the test process. Gas (e.g., air or NO2) may also be added to the fluid to facilitate oxidation and/or testing. Other lubricants, gases and/or additives may also be provided as desired to achieve the desired test conditions for evaluating the test lubricant 102.

The test lubricant 102 is positioned in the test trays 110 in an amount that permits the test lubricant 102 to provide a thin film along the bottom 140. The amount of test lubricant 102 may be such that, when the test trays 110 are tilted in a first position (FIG. 1A), the test lubricant 102 covers the unheated portion of the bottom 140, and when the test trays 110 are tilted in a second position (FIG. 1B), the test lubricant 102 covers the heated portion 142 of the bottom 140.

This configuration allows the system 100 to be positioned at a tilt angle θ₁ so that the lubricant in the test trays 110 will remain in the ambient, non-heated side 144. At a given interval (e.g., about once every minute for a given duration), the cylinder 124 may activate the platform 104 to tilt test block 108 and trays 110 so that the test lubricant 102 moves to the heated portion 142 at the ‘piston’ temperature and at the tilt angle θ₂. This tilting may continue for a given duration (e.g., about 14 hours). The process may be repeated as desired. The portion of the test lubricant 102 that flows between the unheated portion 144 to the heated portion 142 of the test tray may be varied between 0 and 100% by varying the angles θ₁ and θ₂.

In a given example operation, the system 100 is initially positioned as shown in FIG. 1A. The tilt angle θ₁ of the system 100 is in a first position with the first tilt angle θ₁ at about −30 degrees. The heating block 106 is activated to heat the test block 108, and the test trays 110 are positioned on the test block 108 with the test lubricant 102 therein. In the tilted position of FIG. 1A, the test lubricant 102 covers the unheated portion 144 of the test tray 110, but does not extend over the heated portion 142. In this position, the test lubricant 102 is in the unheated portion 144 of the test tray 110 away from the heat received from test block 108.

The system 100 is then movable to a second position at a second tilt angle θ₂ as shown in FIG. 1B. In this position, a portion of the test lubricant 102 flows across the heated portion 142 of the test tray 110 and receives heat applied from the test block 108. The remaining portion of the test lubricant 102 on the unheated portion 144 of the test tray 110 does not receive heat. The system 100 may be selectively tilted between tilt angles of a given duration, as shown in FIGS. 1A and 1B to selectively permit the test lubricant 102 to flow over the heated portion 142 of the test tray 110.

FIGS. 2A1-A4 and 2B depict examples of test trays 110 and 110′ that may be used with the system 100 of FIGS. 1A and 1B. FIG. 2A1-A4 each show one of the test trays 110 as shown in FIG. 1A. FIGS. 2A1-A4 show the progression of deposits that form on the test tray 110 as various tests are performed. For example, these figures may show the same test tray 110 with the same test lubricant 102 with varied test times (e.g., at t₁-t₄, respectively). In another example, these figures may show a different test lubricant 102 in each test tray 110 tested using the same test cycles.

The test trays 110 show deposit patterns 143a-d formed on each of the test trays 110. The deposit patterns 143a-d of each of the test trays 110 may vary depending on the selected test conditions, such as the configuration of the test lubricant 102, the test time, the test heat, etc. The resulting deposits may be visually analyzed and compared to determine performance of the various lubricants. This demonstrates that various test conditions may be performed in one or more of the test trays 110 for comparison.

FIG. 2B shows an alternate test tray 110′ usable with the system 100 of FIGS. 1A and 1B. The test tray 110′ is similar to the test tray 110 and works in a similar manner, except that the test tray 110′ has a different shape with multiple test cavities 248. In this configuration, a single tray 110′ may be used to test one or more lubricants 102. The test tray 110′ may also be secured to the test block 108 by bolts 250. A fastening plate 252 may also be provided to secure the test tray in position. The test tray 110′ may optionally be provided with a lid 254 to cover the cavities 248. As shown, the test tray 110′ has a series of rectangular cavities 248, but any shape may be provided. Combinations of the various test trays 110, 110′ may be provided.

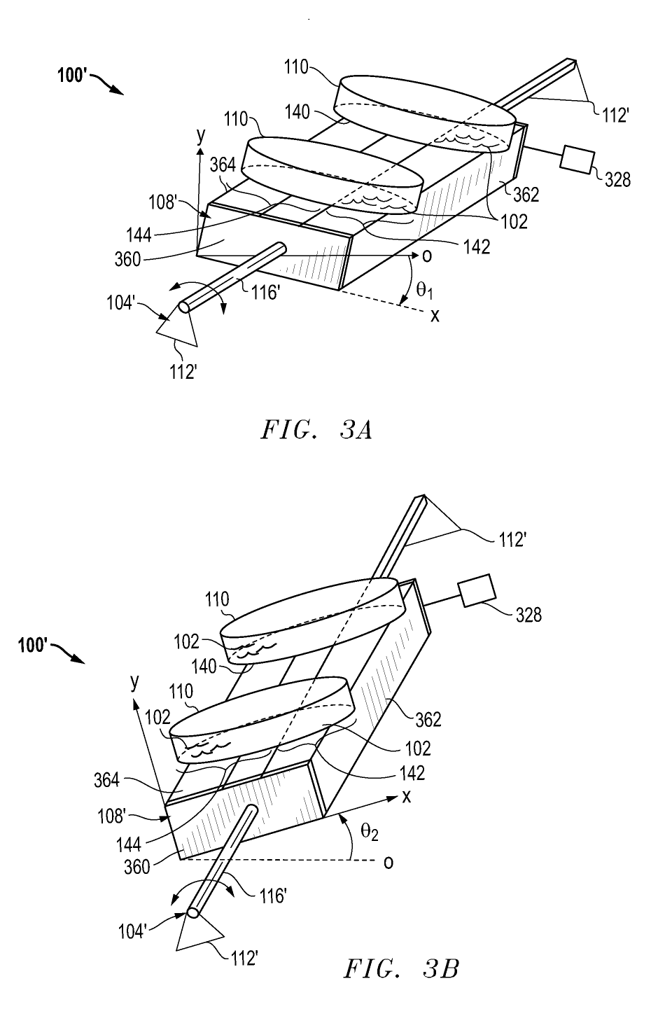

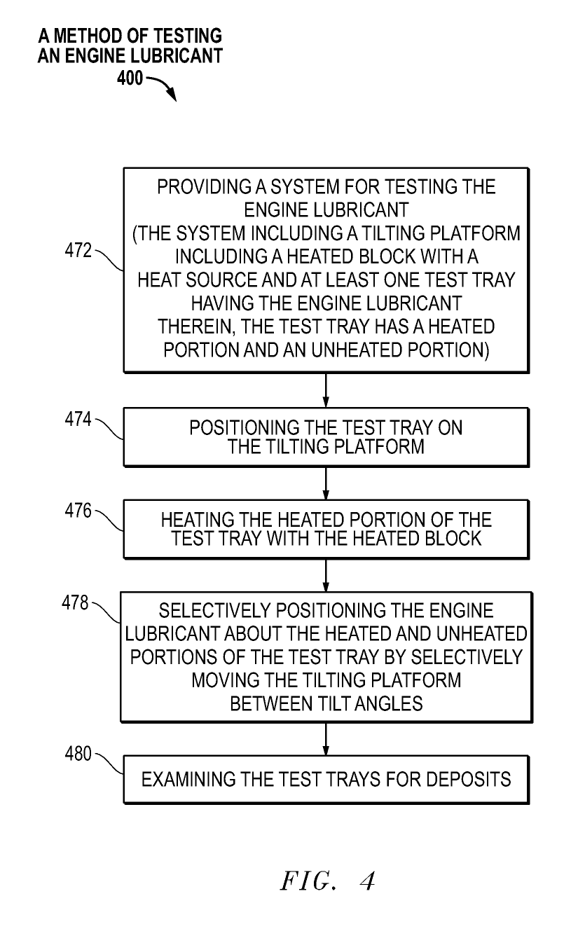

FIGS. 3A and 3B depicts an alternate system 100′ usable for evaluating a lubricant 102. FIG. 3A depicts the system 100′ at a first tilt angle θ₁. FIG. 3B depicts the system 100′ at a second tilt angle θ₂. In this version, the system 100′ includes a platform 104′, an integrated, heated text block 108′ and test trays 110. The platform 104′ includes a pair of supports 112′ positionable on the surface. The integrated heated test block 108′ is rotationally connectable to the supports 112′ via a pivot arm 116′.

The test block 108′ may include a frame 360, a heated bar 362 and an unheated bar 364 supported thereby. The heated bar 362 and the unheated bar 364 are secured in parallel by the frame 360. The pivot arm 116′ may extend through the test block 108′ or include two portions connected to the frame 360 on opposite sides of the test block 108′. A space is provided between the heated bar 362 and the unheated bar 364 to reduce heat transfer therebetween.

The heated bar 362 may be provided with a heat source 328 for applying heat thereto as schematically depicted. The heated bar 362 may be a conductive metal for applying heat to the test trays 110. The unheated bar 364 may be made of a non-conductive material to prevent heat transfer to the test trays 110. The unheated bar 364 may optionally be heated, but may remain at a lower temperature to that of the heated bar 362. The unheated bar 364 may be heated to a temperature less than the temperature of the heated bar 362.

The test trays 110 may be any of the test trays as previously described. The test trays 110 are positioned on the test block 108′ such that heat from heated bar 362 applies heat to a heated portion 142 of the test trays 110. Heat is not applied to the unheated bar 364 or the unheated portion 144 of the test tray 110 resting thereon.

The test block 108′ may be tilted about the pivot arm 116′ at various tilting angles θ′. The test block 108′ may be tilted from a tilted position θ₁ (e.g., about −30 degrees) as shown in FIG. 3A, to a flat position θ₂ (e.g., about −30 degrees) as shown in FIG. 3B. The test block 108′ may be tilted at any angle, such as between from about +30 degrees to about −30 degrees. The movement between tilt angles moves the test lubricant 102 along a bottom 140 of the test trays 110 to provide selective heating. The test may be performed between + and − tilt angles so that the test lubricant 102 may be completely on the unheated portion of the tray at one angle and completely on the heated portion of the tray at another angle. The unheated portion of the test tray 110 may also be heated to a temperature less than the heated portion of the test tray.

FIG. 4 depicts a method 400 of testing an engine lubricant. The method involves 472—providing a system for testing the engine lubricant (the system including a tilting platform including a heated block with a heat source and at least one test tray having the engine lubricant therein, the test tray has a heated portion and an unheated portion), 474—positioning the test tray on the tilting platform, 476—heating the heated portion of the test tray with the heated block, 478—selectively positioning the engine lubricant about the heated and unheated portions of the test tray by selectively moving the tilting platform between tilt angles, and 480—examining the test trays for deposits.

The selectively positioning may involve positioning an unheated portion of the test tray about an unheated block of the heated tilting platform. The selectively moving may involve pivotally moving the platform and/or rotationally moving the platform. The method may be repeated as desired and performed in any order.

While the embodiments are described with reference to various implementations and exploitations, it will be understood that these embodiments are illustrative and that the scope of the inventive subject matter is not limited to them. Many variations, modifications, additions and improvements are possible. For example, one or more test trays with one or more test lubricants may be positioned with unheated and heated portions positioned on a test block as described herein may be used to heat the wellbore.

Plural instances may be provided for components, operations or structures described herein as a single instance. In general, structures and functionality presented as separate components in the exemplary configurations may be implemented as a combined structure or component. Similarly, structures and functionality presented as a single component may be implemented as separate components. These and other variations, modifications, additions, and improvements may fall within the scope of the inventive subject matter.

What is claimed is:

1. A system for testing an engine lubricant, comprising:
a tilting platform selectively movable between tilt angles, the tilting platform comprising a heated block with a heat source; and
at least one test tray having the engine lubricant therein, the at least one test tray having a heated portion and an unheated portion, the at least one test tray tiltably positionable on the tilting platform with the heated portion heatable by the heated block whereby the engine lubricant is selectively positionable about the heated and unheated portions of the at least one test tray.

2. The system of claim 1, wherein the tilting platform comprises a base portion and a tilting portion pivotally connectable by a hinge.

3. The system of claim 1, wherein the tilting platform comprises a pair of supports with a support arm operatively connecting the heated block thereto.

4. The system of claim 1, wherein the tilting platform comprises a test block positionable on the heated block and heatable thereby.

5. The system of claim 1, wherein the heated block comprises a frame with a heated bar and an unheated bar operatively connectable thereto, wherein the unheated portion of the at least one test tray is positionable on the heated bar and the unheated portion of the at least one test tray is positionable on the unheated bar.

6. The system of claim 6, wherein the unheated bar is heated by a heat source to a temperature lower than a temperature of the heated bar.

7. The system of claim 1, further comprising a motor to selectively move the tilting platform between the tilt angles.

8. The system of claim 9, wherein the motor comprise a piston and a cylinder, piston selectively extendable from the cylinder to move the tilting platform between the tilting angles.

9. The system of claim 9, wherein the motor comprises a rotational driver.

10. The system of claim 1, wherein the at least one test tray comprises one of a plurality of test dish, a dish tray having a plurality of test cups, and combinations thereof.

11. A system for testing an engine lubricant, comprising:
a tilting platform selectively movable between tilt angles, the tilting platform comprising at least one test block heatable by a heat source;
at least one test tray having the engine lubricant therein, the at least one test tray having a heated portion and an unheated portion, the at least one test tray tiltably positionable on the tilting platform with the heated portion heatable by the heated block whereby the engine lubricant is selectively positionable about the heated and unheated portions of the at least one test tray; and
a driver operatively connectable to the tilting platform and selectively moving the tilting platform between tilt angles.

12. A method for testing an engine lubricant, comprising:
providing a system for testing the engine lubricant, comprising:
a tilting platform comprising a heated block with a heat source; and
at least one test tray having the engine lubricant therein, the at least one test tray having a heated portion and an unheated portion;
positioning the at least one test tray on the tilting platform;
heating the heated portion of the at least one test try with the heated block;
selectively positioning the engine lubricant about the heated and unheated portions of the at least one test tray by selectively moving the tilting platform between tilt angles; and
examining the at least one test tray for deposits.

13. The method of claim 12, wherein the selectively positioning further comprises positioning the unheated portion of the at least one test tray about the unheated block of the heated tilting platform.

14. The method of claim 12, wherein the selectively moving comprises one of pivotally moving the platform, rotationally moving the platform and combinations thereof.
